(19) United States
(12) Patent Application Publication
(10) Pub. No.: US 2011/0108027 A1
(43) Pub. Date: May 12, 2011

(54) FILTERING DEVICE, METHOD FOR FILTERING OF A FLUID, AND USE OF THE FILTERING DEVICE

(75) Inventor: Martin Kreutz, Konstanz (DE)

(73) Assignee: NYCOMED GMBH, Konstanz (DE)

(21) Appl. No.: 13/002,697

(22) PCT Filed: Jul. 21, 2009
(86) PCT No.: PCT/EP2009/059361

§ 371 Date: Jan. 5, 2011

(30) Foreign Application Priority Data
Jul. 23, 2008 (EP) 08160964.6

(51) Int. Cl. A61M 11/00; B01D 29/01

(52) U.S. Cl. 128/200.23; 210/446; 210/445; 210/808

(57) ABSTRACT

A filtering device comprises a filtering means (8); a first plate-like element (4) disposed on the filtering means (8) and having at least one bore (4a); a second plate-like element (5) disposed below the filtering means (8) and having at least one bore (5a); and a plate-like adapter element (1, 2) disposed on the first plate-like element (4) and having at least one bore (1a, 2a). At least one adapter means (1b, 2b, 3) is disposed on the plate-like adapter element (1, 2) on a side opposite to the first plate-like element (4) and is open to the bore (1a, 2a) of the plate-like adapter element (1, 2). The at least one adapter means (1b, 2b, 3) is adapted for connection with a container containing a fluid to be filtered.

### FIELD OF THE INVENTION

The present invention relates to a filtering device, in particular to a filtering device which is suitable for filtering pharmaceutical preparations having a small volume.

### BACKGROUND OF THE INVENTION

In general, there are two procedural methods for the determination of particulate matter in injections: microscopic procedures and light obscuration procedures (see for instance United States Pharmacopeia USP <788> “Particulate Matter in Injections”). The light obscuration particle count test is applied in case of large-volume injections labeled as containing more than 100 mL, and the microscopic particle count test may be applied to both large-volume and small-volume injections.

The microscopic particle count procedure requires filtration on specific plastic filters under clean room conditions and subsequent counting of particles under a reflected-light microscope. Filtration assemblies which comply with USP-requirements are rather complex in structure and cost-intensive to manufacture, because e.g. the entire filtration assembly has to be arranged in a laminar flow enclosure such as a flow bench. In addition, for the determination of particulate matter at least 25 mL volume is required for filtering the fluid over a suction filter. For injections having smaller volumes, the contents of a corresponding number of containers have to be collected prior to the filtering procedure. However, in the field of pharmaceutical analytics, there exists no generally approved procedure for the detection of particulate matter in medicament-containing solutions which is suitable for test volumes smaller than 25 mL. The results of such tests, i.e. detection of particulate matter, are requested by regulatory authorities in the course of the registration process for a new medicament. Prior art analytics require usually the use of several samples in order to get a minimum volume of 25 if the single sample volume is smaller than 25 mL.

### SUMMARY OF THE INVENTION

Aspects of the invention are defined in claims 1, 12 and 16 below. The dependent claims are directed to optional and preferred features.

It is an object of the present invention to provide a filtering device which is suitable for filtering of test volumes smaller than 25 mL.

It is a further object of the present invention to provide a filtering device which has a simple construction, which can be easily and inexpensively manufactured and is easy to use.

To solve the above objects, in one aspect, the present invention provides a filtering device, comprising: a filtering means; a first plate-like element disposed on the filtering means and comprising at least one bore having a first diameter; a second plate-like element disposed below the filtering means and comprising at least one bore having a second diameter, the second diameter having at least the same size as the first diameter of the bore of the first plate-like element, and wherein the bore of the second plate-like element is disposed substantially centered below the bore of the first plate-like element; a plate-like adapter element disposed on the first plate-like element and comprising at least one bore having a third diameter, the third diameter substantially corresponding to the first diameter of the bore of the first plate-like element, and wherein the bore of the plate-like adapter-element is disposed substantially centered above the bore of the first plate-like element; and at least one adapter means disposed on the plate-like adapter element on a side opposite to the first plate-like element and being open to the bore of the plate-like adapter element, wherein the at least one adapter means is adapted for connection with a container containing a fluid to be filtered.

In the filtering device of the present invention, the filtering means is sandwiched between the first and second plate-like elements which both comprise bores substantially centered above one another. On top of the first plate-like element, the plate-like adapter element is disposed having the bore substantially centered above the bore of the first plate-like element and being open to the adapter means for connection with the container containing the fluid to be filtered. By means of this arrangement, the fluid to be filtered can be concentrated on a very small area on the filtering means, corresponding to the size of the respective bores. In use, particulate matter which is potentially contained in the fluid to be filtered can be collected on this small “spot” on the filtering means. Since only this very small area of the filtering means is actually used for filtering, also fluids having very small volumes may be filtered with the inventive filtering device.

In addition, the inventive filtering device—comprising the filtering means, first and second plate-like elements, the plate-like adapter element and the adapter means—has a simple construction with a small number of parts only and, thus, can easily and inexpensively be manufactured. In use, no additional precautions such as use of a laminar flow enclosure as in the prior art have to be taken, thus, the inventive filtering device cannot only be easily and inexpensively manufactured, but is also easy to use. Suitably, all components of the inventive filtering device are made of an inert material, i.e. a material which does not react with any ingredient contained in the fluid to be filtered. Suitable inert material comprise, but are not limited to high-grade steel, aluminum, PTFE, POM, polyacrylic glass or brass.

In the context of this disclosure, the term “plate-like” is intended to encompass any flat and thin element irrespective of the material composing the element, i.e. any element/material being essentially planar and having a thickness distinctly smaller than its width and length dimensions. Further, the term “fluid” is intended to encompass liquids as well as gases. The expression “fluid to be filtered” relates to any fluid which is to be filtered by means of the inventive filtering device in order to detect particulate matter possibly contained therein.

The first and second plate-like elements and the plate-like adapter element each may comprise a single bore or a plurality of bores, i.e. two or more bores, which are respectively centered on top of each other in the assembled state of the filtering device. If more than one bore in each element is present, several filtering tests can be conducted with one filtering means. Since only the small area of the respective bore is used for the filtering operation, in case of multiple bores the respective area of one bore is easily distinguishable from that of another bore. Thereby, the respective filtering residue (particulate matter) of one sample (fluid to be filtered) cannot be mistaken for the respective filtering residue of another sample. Thus, the provision of more than one bore provides for efficient filtering of multiple samples without the need of disassembling the filtering device and cleaning or replacing the filtering means after each filtering run.

Suitably, the at least one adapter means is formed integrally with the plate-like adapter element. Thereby, less individual parts are required to compose the filtering device, contributing to ease of assembly and inexpensive manufacture.

According to another preferred embodiment, the at least one adapter means is formed separately from the plate-like adapter element. Preferably, the adapter means and plate-like adapter element are designed such that they can be interconnected by means of a screw coupling. In this case, the plate-like adapter element may be provided with a connection bore having a female thread, the connection bore being open to the at least one third-diameter-bore in the plate-like adapter element, and the adapter means may be provided with a male thread. Thereby, the adapter means and plate-like adapter element may easily be screwed together in a fluid-tight manner, thus, allowing for an easy and quick exchange of the adapter means without the necessity to store multiple plate-like adapter elements each having differently shaped adapter means.

Suitably, the adapter means is adapted to be connected to a further adapter means, the further adapter means being disposed between the adapter means and the plate-like adapter element. The further adapter means may either be integrally formed with the plate-like adapter element or may be formed separately from the plate-like adapter element. In case the further adapter means is formed separately from the plate-like adapter element, the two parts may for example be interconnected by means of a screw coupling, as outlined above. This preferred embodiment comprising the adapter means, which is formed separately from the plate-like adapter element, and the further adapter means, which may either be formed in one piece with the plate-like adapter element or separately from the plate-like adapter element, is a so-called “adapter-in-adapter” solution. If an adapter means being shaped for connection with a different container is needed, one adapter means can easily be placed in the other adapter means, allowing for more flexibility in use of the inventive filtering device.

Both adapter means are preferably connected in a fluid-tight manner so as to prevent any loss of a sample to be filtered. The fluid-tight connection between the two adapter means can be achieved in any suitable fashion, e.g. by means of a screw coupling, interference fit or the provision of additional sealing elements, such as e.g. an O-ring provided around the outer periphery of the adapter means and/or the inner periphery of the further adapter means. If the fluid-tight connection of the two adapter means is to be achieved via an interference fit, preferably at least one of the two adapter means is composed of a plastic material, such as Teflon®. However, the material is not limited to Teflon®, rather any suitable inert plastic material may be used.

According to one preferred embodiment, at least one of the first plate-like element and the second plate-like element comprises at least one sealing element facing the filtering means, said sealing element being disposed around the respective bore such that the first plate-like element and the second plate-like element, with the filtering means sandwiched in between, constitute a sealed unit around the respective bore. By means of the at least one sealing element a sealed unit can be obtained, thus, loss of fluid can be prevented and it can be assured that all of the fluid at first passes through the bore of the first plate-like element, then through the filtering means and finally through the bore of the second plate-like element. As a sealing element, for instance an O-ring may be provided in at least one of the first and second plate-like elements, preferably disposed in a groove formed in the respective plate-like element(s).

Preferably, the at least one sealing element is formed integrally with at least one of the first plate-like element and the second plate-like element. In this regard, it is contemplated that the at least one sealing element may be realized in form of one or more lips surrounding the respective bore. Due to the integral provision of the sealing element(s) with the plate-like element(s), less individual parts are necessary, thus, simplifying the assembly and manufacture of the filtering means. If the sealing element is formed integrally with the plate-like element(s), the respective plate-like element is preferably made of plastics, such as Teflon®.

Suitably, the first diameter of the bore of the first plate-like element and the third diameter of the bore of the plate-like adapter element are smaller than 25 mm, preferably smaller than 10 mm, more preferably smaller than 5 mm, most preferably smaller than 3 mm. Because of the small diameters of the respective bores, also small volumes of fluid may be filtered and effectively concentrated on the small spot of the filtering means defined by the size of the respective bore.

Suitably, the second diameter of the bore of the second plate-like element is larger than the first diameter of the bore of the first plate-like element, preferably 5% larger, more preferably 10% larger. Thereby, easy outflow of the fluid having passed the filtering means is promoted and it can be assured that no fluid will be left on the filtering means.

Preferably, the filtering device further comprises a base plate disposed below the second plate-like element, wherein the base plate comprises at least one bore having a fourth diameter, the fourth diameter having at least the same size as the second diameter of the bore of the second plate-like element, and wherein the bore of the base plate is disposed substantially centered below the bore of the second plate-like element. If a base plate is present, the remaining components of the filtering device are disposed on the base plate, constituting a filtering unit comprising the base plate, first and second plate-like elements, filtering means, plate-like adapter element and adapter means. The base plate may be fixed to the other components of the filtering device, preferably to the plate-like adapter element, so that all components constituting the filtering device are kept in close contact with each other, constituting a fluid-tight unit. Fixing of the base plate to the other components of the filtering device may be achieved by using a suitable clamping means such as a screw clamp. Preferably, the base plate is fixed to the plate-like adapter element by means of a screw connection. In this regard, it is preferred that at least two holes having female threads are provided in the base plate and at least two through-holes are provided in the plate-like adapter element, wherein the at least two through-holes substantially correspond in position to the at least two threaded holes in the base plate. Having such a construction, a screw may be passed through each respective through-hole in the plate-like adapter element, screwed into and tightened in each respective threaded hole in the base plate, to keep the parts constituting the filtering device in close contact with each other. However, alternatively, if no base plate is provided, the respective parts constituting the filtering device may be manually hold in close contact with each other during the filtering operation. To aid in keeping the respective parts constituting the filtering device suitably aligned to each other, the plate-like adapter element, first plate-like element and preferably also second plate-like element may be respectively provided with at least one guiding groove, formed on one element, and at least one corresponding guiding projection engaging the guiding groove, formed on the other element. Further, it is contemplated that the plate-like adapter element may be directly fixed to the second plate-like element, e.g. by means of a screw connection or a clamping means.

Preferably, the plate-like adapter element comprises at least one sight opening. By means of the sight opening any leakage of fluid in the contact area between the plate-like adapter element and the first plate-like element can be optically observed. Thus, the at least one sight opening is a simple yet very effective means to detect possible leakages of the fluid being filtered.

Suitably, the filtering device further comprises a sealing member, preferably an O-ring, disposed on the adapter means for connection with a container containing a fluid to be filtered. Through the provision of a sealing member an the adapter means, fluid-tight connection between the adapter means and container can easily and effectively be achieved.

In one preferred embodiment, a support element for supporting the filtering means is disposed at least partially in the area of the bore of the second plate-like element. The support element may be formed integrally with the plate-like element or may be provided as a separate part. The support element enhances the stability of the filtering means in the area of the bore of the second plate-like element. Thereby, even if a fluid to be filtered is pressed through the filtering device with considerable force, damage of the filtering means, e.g. the filtering means getting “baggy”, can be prevented. Preferably, the support element is composed of a material allowing the fluid being filtered to pass through easily, such as a filtering material, preferably a frit or a grid-like inert supporting material.

In a second aspect, the present invention provides a method for filtering of a fluid, comprising the steps of disposing a dispensing opening of a container containing the fluid to be filtered on the adapter means of the inventive filtering device; and actuating a dispensing means of the container so as to press a single dose of the fluid contained therein into the filtering device. In the inventive method, a single dose of the fluid to be filtered is pressed through the inventive filtering device. Since the fluid is pressed through the filtering device, it is not necessary to apply a vacuum for filtering of the fluid. Thereby, also small volumes of fluid may be filtered. In addition, such a filtering operation can be easily carried out by simply disposing the dispensing opening of the respective container at the adapter means and actuating the dispensing means so as to dispense a single dose of the fluid to be filtered into the filtering device.

The inventive method is carried out with a filtering device which is easy to assemble and use, without requiring special precautions such as a clean room environment and/or a laminar flow enclosure. Advantageously, the method according to the present invention reduces potential cross-contamination with particles in the environmental surroundings, reduces significantly the manual effort in conducting a filtering operation, reduces significantly the costs per test run to be conducted and produces very reproducible results. Further, the sensitivity of the determination of the content of particulate matter in the respective fluid, even for very small volumes of fluid, can be enhanced significantly compared to methods of the prior art.

In a preferred embodiment, the single dose comprises a volume of less than 25 ml, preferably less than 10 mL, more preferably less than 500 μL, most preferably less than 100 μL. According to the present invention, even single doses having a very small volume may be filtered, the single doses suitably containing less than 100 mL, 50 mL, 25 mL, 20 mL, 10 mL, 5 mL, 1 mL, 500 μL or 100 μL, respectively. Since the residue of the fluid being filtered is collected on a small spot of the filtering means defined by the size of the bores of the respective parts of the filtering device, even very small volumes of fluid may be effectively and reliably filtered.

In another preferred embodiment, the container comprises a nasal spray having a nasal-adapter, a syringe having a Luer's cone or a MDI (metered dose inhaler) having a valve tube, and wherein the nasal-adapter, Luer's cone or valve tube, respectively, of the container is disposed on the adapter means. In general, the inventive filtering method can be applied to any kind of container capable of dispensing a single dose of a fluid to be filtered. With the inventive method, also a determination of particulate matter in fluids contained in inhalers, e.g. with a liquid gas propellant, as well as in gaseous pharmaceutics, e.g. narcotics, is possible. The respective dispensing outlet of the container has to be placed on the adapter means, which in turn has to be correspondingly shaped to receive the dispensing outlet. Since the inventive method is not restricted to a specific type of container, it can be considered an “all-purpose method” leaving a high degree of freedom in the field of preparation for sample analysis.

Preferably, the adapter means comprises a recess having a shape corresponding to the nasal adapter, Luer's cone or valve tube, respectively, and wherein the nasal adapter, Luer's cone or valve tube, respectively, is disposed in the recess of the adapter means. The shape of such a recess can be easily adapted to correspond to the shape of a respective dispensing outlet, such as nasal adapter, Luer's cone or valve tube. In use, the dispensing outlet can be matingly engaged with the adapter means via the recess so as to provide a fluid-tight connection between the two elements.

In a third aspect, the present invention provides use of the inventive filtering device for sample preparation for determination of particle-content of pharmaceutical preparations, preferably parenteral preparations, liquid preparations for cutaneous application, nasal preparations, and preparations for inhalation. In the context of the present application, these different kinds of pharmaceutical preparations are to be interpreted in accordance with the definitions given in the European Pharmacopoeia V6.01, 1/2008. The inventive use of the filtering device applies to all kinds of pharmaceutical preparations and, thus, a high degree of freedom in sample preparations for determination of particle-content is achieved. Further, since the inventive filtering device is suitable for filtering of fluids having very small volumes, the filtering device is particularly useful for the filtering/sample preparation of high-priced test fluids, such as fluids containing proteins.

### BRIEF DESCRIPTION OF THE DRAWINGS

For a better understanding of the present invention, and to show more clearly how the same may be carried into effect, reference will now be made, by way of example only, to the accompanying drawings, in which:

FIG. 1 is an exploded view schematically illustrating a filtering device according to a first embodiment of the present invention;

FIG. 2 is a perspective view showing the individual components of the filtering device according to the first embodiment, wherein two plate-like adapter elements having different adapter means are shown;

FIG. 3 is a perspective view showing the two plate-like adapter elements of FIG. 2 in more detail, wherein FIG. 3a shows an adapter element having an adapter means for a nasal spray with a nasal-adapter, and FIG. 3b shows an adapter element having an adapter means for a syringe with a Luer's cone;

FIG. 4 is a plan view showing the plate-like adapter element of FIG. 3a in more detail;

FIG. 5 is an exploded view schematically illustrating a filtering device according to a second embodiment of the present invention; and

FIG. 6 is a schematic view showing the adapter means of FIG. 5 in more detail, wherein FIG. 6a is a perspective view of the adapter means, and FIG. 6b is a sectional view of the adapter means.

### DETAILED DESCRIPTION OF PREFERRED EMBODIMENTS

In the following, preferred embodiments of the present invention will be described in detail with reference to the accompanying drawings.

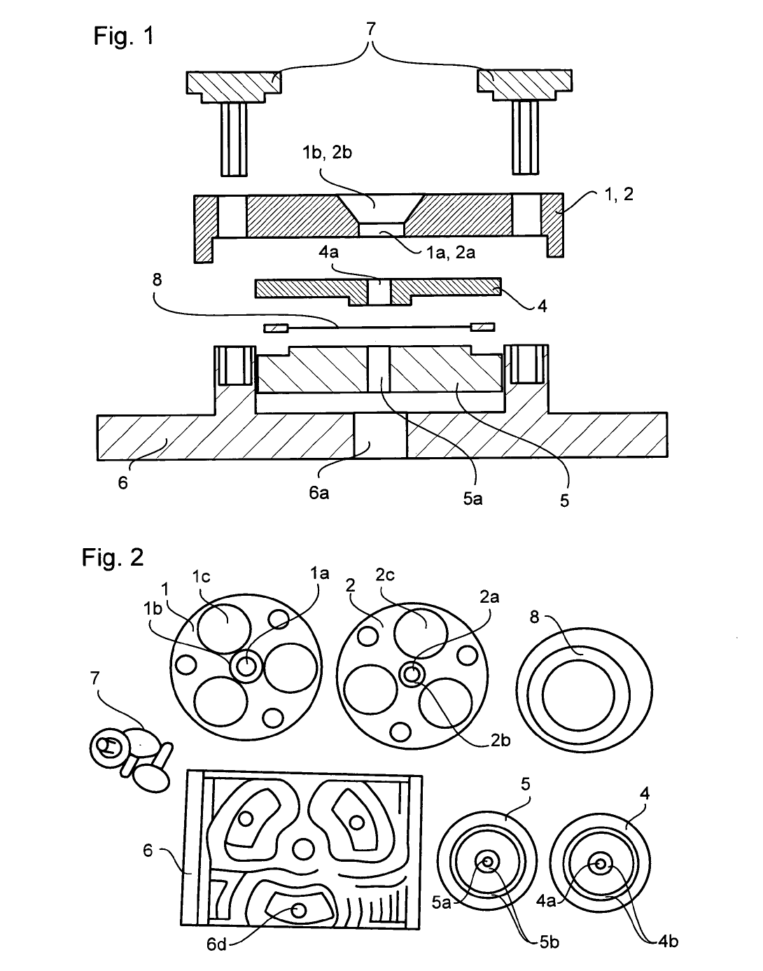

FIG. 1 is a schematic view illustrating a filtering device according to a first embodiment of the present invention. The filtering device comprises a filtering means 8, first and second plate-like elements 4, 5, and a plate-like adapter element 1, 2.

The first plate-like element 4 is disposed on top of the filtering means 8 and comprises a bore 4a. The bore 4a has a diameter (first diameter) which can be varied depending on the particular intended use. As regards the size of the diameter, bores having a diameter between approximately 1 mm and approximately 3 mm are considered suitable for volumes of fluid to be filtered of less than approximately 1 ml. For a range of volume between approximately 1 ml and approximately 5 ml, bores having a diameter between approximately 3 mm and approximately 5 mm are considered suitable. For larger volumes of fluid to be filtered, bores having a diameter up to approximately 25 mm, preferably approximately 10 mm, are considered suitable.

As shown in FIG. 1, the second plate-like element 5 is disposed below the filtering means 8 and comprises a bore 5a. The diameter (second diameter) of the bore 5a is at least the same size as the first diameter of the bore 4a of the first plate-like element 4 and, therefore, may also vary depending on the particular intended use, as outlined above. In the assembled state of the filtering device, the bore 5a of the second plate-like element 5 is disposed substantially centered below the bore 4a of the first plate-like element 4.

The first and second plate-like elements 4, 5 can be made of any suitable material, preferably an inert material, i.e. a material not reacting with any ingredient contained in the fluid to be filtered. Examples of suitable materials for the first and second plate-like elements 4, 5 comprise, but are not limited to Teflon®, e.g. Teflon-PTFE, Teflon-PFA, low density polyethylene (LDPE), high density polyethylene (HDPE), polyamide, acrylonitrile butadiene styrene (ABS), polyoxymethylene (POM), e.g. Delrin® available from DuPont, or aromatic polyamides or polyaramids in short, e.g. Kevlar® available from DuPont.

The filtering means 8 can comprise any suitable filtering means, as long as it can be sandwiched between the first and second plate-like elements 4, 5. Preferably the filtering means 8 comprises a plane, essentially two-dimensionally extending filtering material. In the context of this disclosure, the term “essentially two-dimensionally extending filtering material” relates to a material which is considerably larger in the width and length dimensions than in the height dimension, for example with a ratio of width/length dimension to height dimension of at least 10:1. Preferably the ratio of width/length dimension to height dimension is at least 100:1, more preferably at least 500:1, most preferably at least 1000:1. Preferably, the filtering means 8 has a pore size smaller than 10 μm, more preferably smaller than 5 μm, most preferably smaller than 1 μm. A suitable filtering means 8 may comprise, but is not limited to, a gold filter (e.g. gold-plated plastic material), fiberglass (micro-)filter, frit, or a filtering material comprising cellulose nitrate, polytetrafluoroethylene (PTFE) or nylon. Use of a gold filter has the advantage that such filters may be stored in a space-saving manner for a long time without deterioration. A variety of filtering means, which are considered suitable for the filtering device of the present application, are commercially available (e.g. from RAP-ID, Whatman plc, Macherey-Nagel GmbH), such as:
gold filters having a diameter of 29 mm and a pore size 0f 0.45, 0.8 or 3.0 μm, respectively;
fiberglass microfilters having a diameter of 21, 24, 25 or 37 mm, respectively, and a pore size of 1.6 μm;
filters made of cellulose nitrate having a diameter of 47 or 50 mm, respectively, and a pore size of 0.2 or 0.45 μm, respectively; and
filters made of PTFE having a diameter of 25, 47 or 50 mm, respectively, and a pore size of 0.2, 0.45, 1.2 or 5 μm, respectively.

As shown in FIG. 1, the plate-like adapter element 1, 2 is disposed on the first plate-like element 4 and comprises a bore 1a, 2a. The diameter (third diameter) of the bore 1a, 2a corresponds substantially to the diameter (first diameter) of the bore 4a of the first plate-like element 4. In the assembled state, the bore 1a, 2a of the plate-like adapter element 1, 2 is disposed substantially centered above the bore 4a of the first plate-like element 4. The plate-like adapter element 1, 2 can be made of any suitable material, depending on the respective demanded inert properties. This means, any suitable material may be used as long as no interaction between the fluid to be filtered and the plate-like adapter element 1, 2 occurs. Suitable materials for the plate-like adapter-element 1, 2 comprise, but are not limited to, high-grade steel, aluminum, PTFE, POM, polyacrylic glass or brass. Preferably, the respective materials for the plate-like adapter-element 1, 2 should allow processing on a lathe or should be moldable by means of injection moulding, so as to keep the manufacturing costs at a reasonable level.

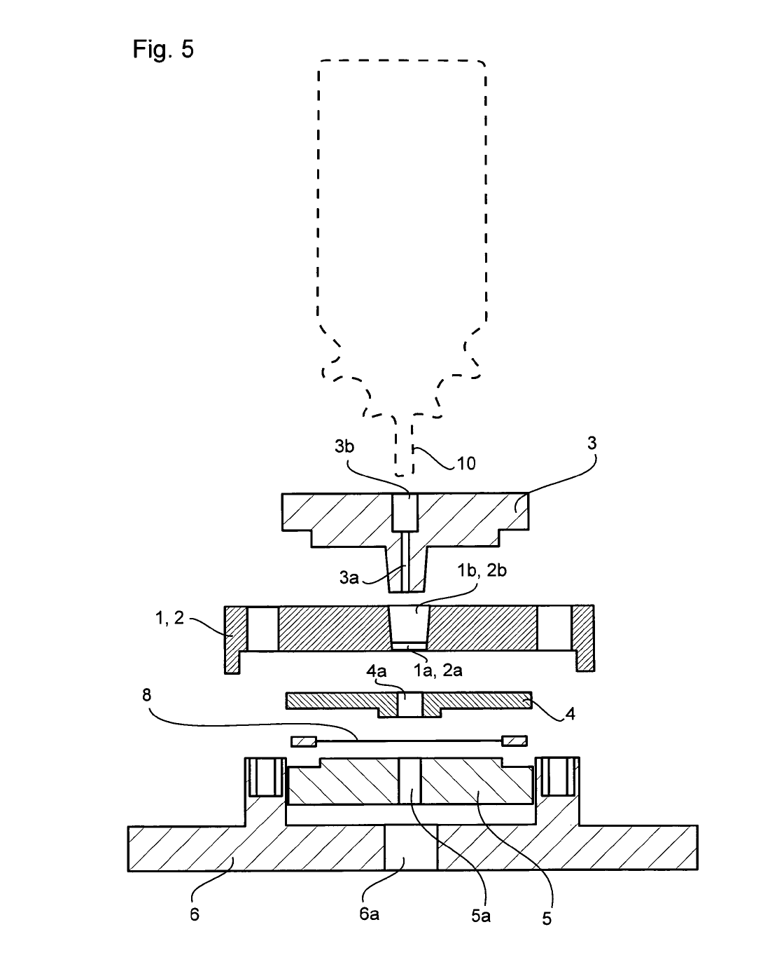

An adapter means 1b, 2b is disposed on the plate-like adapter element 1, 2 on a side opposite to the first plate-like element 4. The adapter means 1b, 2b is open to the bore 1a, 2a of the plate-like adapter element 1, 2, and is adapted for connection with a container containing a fluid to be filtered. This means, the adapter means 1b, 2b has a size and shape such that a respective dispensing outlet of a container may be disposed on the adapter means 1b, 2b for conducting a filtering process to be described further below. Through the provision of the adapter means 1b, 2b, different types of containers may be used for carrying out the filtering operation. The size and shape of the adapter means 1b, 2b depends on the specific container to be used for the filtering process. The type of container is not restricted to a specific type; generally all containers—from which a single dose of a fluid may be dispensed—are suitable to conduct the filtering process. Containers containing a propellant may be used as well as containers in form of pump bottles. Preferably, the container comprises a nasal spray having a nasal adapter, a syringe having a Luer's cone or a MDI having a valve tube 10 (see FIG. 5). In the context of the present disclosure, “nasal adapters” of nasal sprays are adapters which are suitable to be placed in the nostril of a patient for dispensing, in use, a single dose of the fluid in the container into the nasal cavities of the patient.

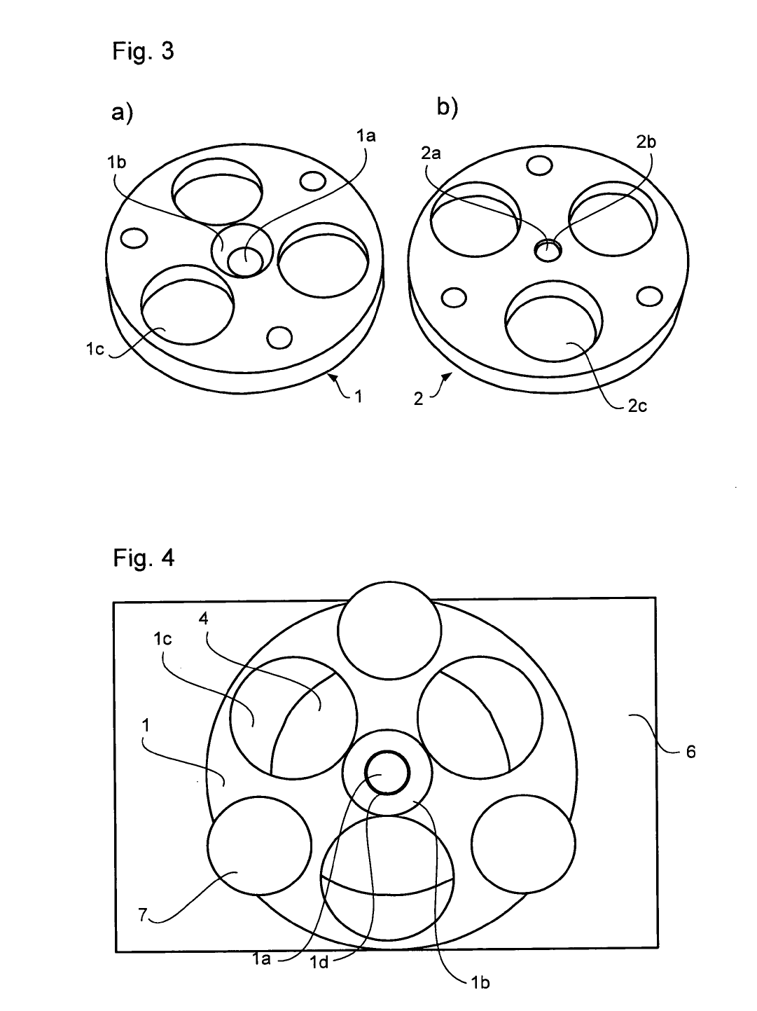

As can be seen from FIG. 1, in the present embodiment, the adapter means 1b, 2b is formed integrally with the plate-like adapter element 1, 2 in form of a recess in the plate-like adapter element 1, 2. The recess has a shape corresponding to the nasal adapter (see FIG. 3a)), Luer's cone (see FIG. 3b)) or valve tube 10, respectively. However, the adapter means 1b, 2b may also be formed separately from the plate-like adapter element 1, 2 and may for instance be connected with the adapter element 1, 2 via a screw coupling (not shown).

In the embodiment shown in FIG. 1, the filtering device further comprises a base plate 6 disposed below the second plate-like element 5. The base plate 6 comprises a bore 6a having a diameter (fourth diameter) of at least the same size as the diameter (second diameter) of the bore 5a of the second plate-like element 5. As can be taken from FIG. 1, the bore 6a of the base plate 6 is disposed substantially centered below the bore 5a of the second plate-like element 5. The plate-like adapter element 1, 2 is fixed to the base element 6 by means of screws 7 passing through respective through-holes 1d, 2d (see FIG. 2) in the plate-like adapter element 1, 2 and being screwed into and tightened in correspondingly arranged bores 6d (see FIG. 2) having female threads in the base plate 6. By means of the tightened screw connection, a fluid-tight unit at least among the plate-like adapter element 1, 2, the filtering means 8 and the first and second plate-like elements 4, 5 is achieved. In the context of the present application, the term “fluid-tight unit” encompasses a unit which is liquid-tight and/or gas-tight, i.e. impermeable or at least substantially impermeable to liquids and/or gases.

FIG. 2 is a perspective view showing the individual components of the filtering device according to the first embodiment, wherein two plate-like adapter elements 1, 2 having differently shaped adapter means 1b, 2b are shown. The adapter means 1b is shaped for connection with a nasal adapter of a nasal spray (not shown), and the adapter means 2b is shaped for connection with a Luer's cone of a syringe (not shown). As can be seen in FIG. 2, each plate-like adapter element comprises three sight openings 1c. By means of these sight openings it can be optically checked during the filtering process if any liquid being filtered escapes laterally into the area between the plate-like adapter element 1, 2 and the first plate-like element 4, thus, not taking part in the filtering process.

In the present embodiment, the first and second plate-like elements 4 and 5 each comprise two sealing elements 4b, 5b in form of sealing lips provided around the respective opening (bore) 4a, 5a on one side of the respective plate-like element 4, 5. The sealing elements are formed integrally with the first plate-like element 4 and the second plate-like element 5. In the assembled state of the filtering device, each of these sealing elements 4b, 5b is disposed so as to face the filtering means 8, either from above or from below, whereby the filtering means 8 is sandwiched in between the first and second plate-like elements 4, 5, constituting a sealed unit around the respective bore 4a, 5a. In the embodiment shown in FIG. 2, the plate-like adapter element 1, 2 and the base plate 6 are made of high-grade steel, the filtering means 8 comprises a gold filter supported by a frame made of plastics, and the first and second plate-like elements 4, 5 are made of Teflon®. In addition, the base plate 6 comprises several ridges or protruding elements 6e for fixing the second plate-like element 5 in position relative to the base plate 6.

FIG. 3 is a perspective view showing the two plate-like adapter elements 1, 2 of FIG. 2 in more detail, wherein FIG. 3a shows an adapter element 1 having an adapter means 1b for a nasal spray with a nasal-adapter, and FIG. 3b shows an adapter element 2 having an adapter means 2b for a syringe with a Luer's cone. As can be seen from FIG. 3a, the adapter means 1b comprises a substantially conically shaped recess corresponding in dimension to a nasal adapter of a nasal spray. Further, as shown in FIG. 3b, the adapter means 2b comprises a conically shaped recess corresponding in dimension to a Luer's cone of a syringe. Both plate-like adapter elements 1, 2 have a circular shape and, thus, can be easily manufactured on a lathe, keeping the manufacturing costs low. However, the shape of the plate-like adapter element 1, 2 is not limited to the circular shape and any suitable shape, e.g. circular, oval, quadrangular or rectangular, may be employed. In this preferred embodiment, the filtering device also comprises a sealing member 1d in form of an O-ring, which is disposed on the adapter means 1b, 2b for fluid-tight connection with a container containing a fluid to be filtered (see FIG. 4).

FIG. 5 is a schematic view illustrating a filtering device according to a second embodiment of the present invention. In the second embodiment, like reference numerals designate like parts as in the first embodiment. The filtering device of the second embodiment corresponds in large parts to the filtering device of the first embodiment. In particular, the filtering device shown in FIG. 5 also comprises a filtering means 8, first and second plate-like elements 4, 5 and a base plate 6. These parts correspond in essence to those described above with regard to the first embodiment and for sake of brevity a detailed description thereof will be omitted.

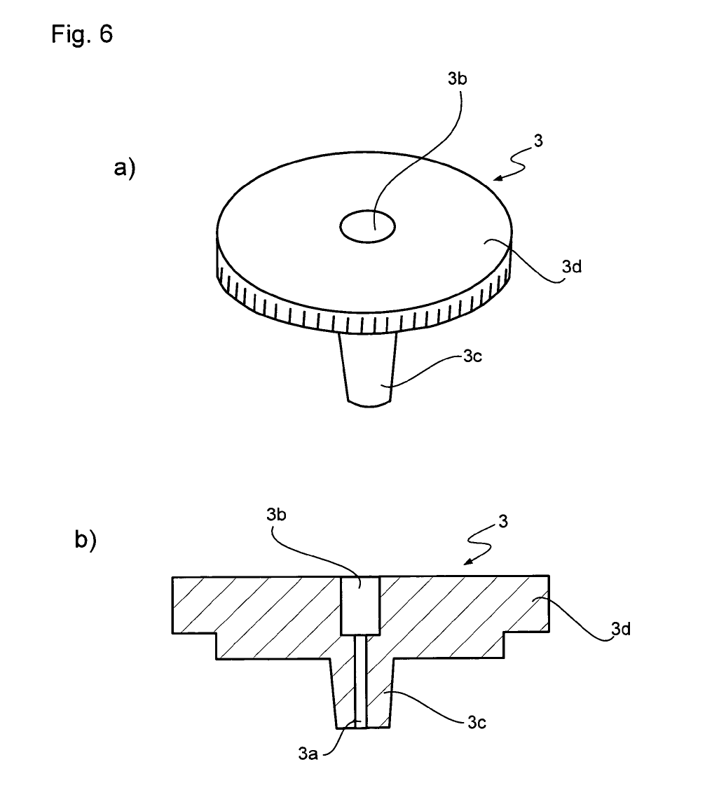

Contrary to the filtering device of the first embodiment, the filtering device shown in FIG. 5 is a so-called “adapter-in-adapter” configuration. As can be seen in the Figure, the filtering device comprises a plate-like adapter element 1, 2 having a bore 1a, 2a. The adapter means 3 is formed separately from the plate-like adapter element 1, 2 and is fitted in a further adapter means 1b, 2b provided integrally with the plate-like adapter element 1, 2. In the second embodiment, the adapter means 3 comprises a recess 3b and a through-hole 3a, as shown in FIGS. 6a and 6b in more detail. The recess 3b is shaped for mating connection with a valve tube 10 (see FIG. 5) of an MDI-container (shown in phantom in FIG. 5). The shape of the recess 3b, however, is not limited to this specific shape, but may e.g. be shaped to allow for mating connection with a Luer's cone of a syringe or a nasal adapter of a nasal spray.

A bottom part 3c of the adapter means 3 is shaped so as to correspond to the shape of the further adapter means 1b, 2b to allow for mating engagement of both adapter means 1b, 2b, 3. Both adapter means 1b, 2b, 3 should be engaged in a fluid-tight manner so as to prevent any possible leakage of the sample being filtered with the filtering device in between the area of the two adapter means 1b, 2b, 3. In addition, the dispensing outlet of a container containing a fluid to be filtered, e.g. the valve tube 10 of the MDI-container, should be engaged in a fluid-tight manner with the adapter means 3 to prevent any fluid leakage.

A top part 3d of the adapter means 3 is preferably formed circularly so that the adapter means 3 may be produced on a lathe for reasons of cost-effectiveness (see FIG. 6a). However, the shape of the top part 3d of the adapter means 3 is not restricted to the circular shape shown in FIG. 6a, and any suitable shape, such as circular, oval, quadrangular or rectangular, may be employed.

In the embodiment shown in FIGS. 5-6, the further adapter means 1b, 2b is provided integrally with the plate-like adapter element made of high-grade steel, and the adapter means 3 is made of plastics, such as Teflon®, to allow for a fluid-tight connection of the two adapter means 1b, 2b, 3 by means of an interference fit. However, the fluid-tight connection between the two adapter means 1b, 2b, 3 may also be achieved by other means such as a screw coupling or the provision of additional sealing elements, e.g. O-rings, provided around the outer periphery of the adapter means 1b, 2b and/or the inner periphery of the further adapter means 3.

Both adapter means are preferably connected in a fluid-tight manner so as to prevent any loss of a sample to be filtered. If the fluid-tight connection of the two adapter means is to be achieved via an interference fit, preferably at least one of the two adapter means is composed of a plastic material, such as Teflon®. However, the material is not limited to Teflon®, rather any suitable inert plastic material may be used.

The filtering device of the present invention may also be provided as a kit of parts. For instance, such a kit of parts may comprise the filtering means 8, the first and second plate-like elements 4, 5, and two or more plate-like adapter elements 1, 2 each having an integrated adapter means 1b, 2b suitable for mating connection with a different type of container. Suitably, the kit of parts comprises only one plate-like adapter element 1, 2 and at least two different types of adapter means 1b, 2b provided separately from the plate-like adapter element 1, 2. Preferably, the at least two adapter means 1b, 2b and the plate-like adapter element 1, 2 are shaped for mating connection via a screw coupling. Thereby, the respective adapter means 1b, 2b may be exchanged easily as the need arises. Still further, such a kit of parts suitably comprises one adapter plate 1, 2 having an adapter means (further adapter means) 1b, 2b and at least two individual adapter means 3 formed separately from the adapter plate 1, 2. Preferably, the at least two adapter means 3 are made of plastics, keeping the costs for the kit of parts at a reasonable level. Any such kit of parts may additionally comprise a base plate 6 and fixing means, such as screws 7, so that all components constituting the filtering device may be kept in close contact with each other to constitute a fluid-tight unit.

The filtering device of the present invention can easily be assembled and disassembled, and the respective adapter means 1b, 2b, 3 can easily be interchanged, so as to adapt the filtering device to different types of containers. After a filtering process has been carried out, the filtering device may easily be disassembled and the respective constituent parts may be cleaned and/or replaced.

In use, a dispensing outlet (dispensing opening) of a container containing the fluid to be filtered is disposed on the respective adapter means 1b, 2b, 3 of the filtering device. Then, a dispensing means such as a piston of a MDI-container or syringe is actuated, so as to press a single dose of the fluid contained therein into the filtering device. With reference to FIG. 5, the valve tube 10 (dispensing outlet) of the MDI-container is placed in the recess 3b provided in the adapter means 3. Then the MDI-container is actuated so as to dispense a single dose of the fluid contained therein. The single dose of the fluid is pressed through the filtering device, leaving particles possibly contained in the fluid on a small spot on the filtering means 8. Similarly, fluid can be drawn into a syringe and subsequently be pressed through the filtering device, or fluid contained in a nasal spray may be pressed through the filtering device. In any case, the fluid to be filtered has to be pressed through the filtering device, which can be solely manually achieved as in the case of actuating a syringe, or aided by a propellant as in the case of a MDI-container. Since the fluid is pressed through the filtering device by actuating the respective container disposed on the adapter means 1b, 2b, 3, it is not necessary to apply a vacuum for the filtering operation. Very small volumes of liquid may also be sucked through the filtering means via capillar action. Capillary forces become effective if a suitable, absorbent material is disposed on the lower side of the filtering means 8, i.e. on the side of the filtering means 8 facing the second plate-like element 5. As the absorbent material any suitable material may be used. Preferably, paper is used as the absorbent material. Suitably, the absorbent material has a size corresponding to the diameter of the bore 5a in the second plate-like element 5, preferably the absorbent material being larger than the diameter of the bore 5a.

As mentioned above, according to the present invention, the fluid is pressed through the filtering means 8 instead of being sucked through the filtering means 8 as in the prior art methods. In addition, the area of the filtering means 8 actually involved in the filtering process is distinctly reduced compared to USP-specifications, e.g. approximately by a factor of 100 in case of a diameter of the respective bores of approximately 3 mm. The inventive filtering device allows for fluid-tight connection between the container with the fluid to be filtered and the very small filtering area of the filtering means 8. Due to the fluid-tight connection, it is not necessary to conduct filtering experiments under a flow bench as in the prior art methods. In the filtering device of the present invention, merely the contact areas between the filtering device and the container have to be free of particles to avoid contamination.

After being pressed through the filtering device, particles possibly contained in the fluid are collected on the very small spot of the filtering means, as described above. For subsequent analysis, the number of the particles may be counted using a commercially available microscope. In addition, further analysis of the particles can be conducted as the need arises. For instance, if a gold filter is used as the filtering material 8, the filter with the particles thereon may be placed in a scanning electron microscope (SEM) and SEM-analysis may be performed.

It will be understood that the present disclosure is for the purpose of illustration only and the invention extends to modifications, variations and improvements thereto within the scope of the appended claims.

1. A filtering device, comprising:
a filtering means (8);
a first plate-like element (4) disposed on the filtering means (8) and comprising at least one bore (4a) having a first diameter;
a second plate-like element (5) disposed below the filtering means (8) and comprising at least one bore (5a) having a second diameter, the second diameter having at least the same size as the first diameter of the bore (4a) of the first plate-like element (4), and wherein the bore (5a) of the second plate-like element (5) is disposed substantially centered below the bore (4a) of the first plate-like element (4);
a plate-like adapter element (1, 2) disposed on the first plate-like element (4) and comprising at least one bore (1a, 2a) having a third diameter, the third diameter substantially corresponding to the first diameter of the bore (4a) of the first plate-like element (4), and wherein the bore (1a, 2a) of the plate-like adapter-element (1, 2) is disposed substantially centered above the bore (4a) of the first plate-like element (4); and
at least one adapter means (1b, 2b, 3) disposed on the plate-like adapter element (1, 2) on a side opposite to the first plate-like element (4) and being open to the bore (1a, 2a) of the plate-like adapter element (1, 2), wherein the at least one adapter means (1b, 2b, 3) is adapted for connection with a container containing a fluid to be filtered.

2. The filtering device according to claim 1, wherein the at least one adapter means (1b, 2b) is formed integrally with the plate-like adapter element (1, 2).

3. The filtering device according to claim 1, wherein the at least one adapter means (3) is formed separately from the plate-like adapter element (1, 2).

4. The filtering device according to claim 3, wherein the adapter means (3) is adapted to be connected to a further adapter means (1b, 2b), the further adapter means (1b, 2b) being disposed between the adapter means (3) and the plate-like adapter element (1, 2).

5. The filtering device according to claim 1, wherein at least one of the first plate-like element (4) and the second plate-like element (5) comprises at least one sealing element (4b, 5b) facing the filtering means (8), said sealing element (4b, 5b) being disposed around the respective bore (4a, 5a) such that the first plate-like element (4) and the second plate-like element (5), with the filtering means (8) sandwiched in between, constitute a sealed unit around the respective bore (4a, 5a).

6. The filtering device according to claim 5, wherein the at least one sealing element (4b, 5b) is formed integrally with at least one of the first plate-like element (4) and the second plate-like element (5).

7. The filtering device according to claim 1, wherein the first diameter of the bore (4a) of the first plate-like element (4) and the third diameter of the bore (1a, 2a) of the plate-like adapter element (1, 2) are smaller than 25 mm.

8. The filtering device according to claim 1, wherein the second diameter of the bore (5b) of the second plate-like element (5) is larger than the first diameter of the bore (4b) of the first plate-like element (4).

9. The filtering device according to claim 1, further comprising a base plate (6) disposed below the second plate-like element (5), wherein the base plate (6) comprises at least one bore (6a) having a fourth diameter, the fourth diameter having at least the same size as the second diameter of the bore (5a) of the second plate-like element (5), and wherein the bore (6a) of the base plate (6) is disposed substantially centered below the bore (5a) of the second plate-like element (5).

10. The filtering device according to claim 1, further comprising a sealing member (1d), disposed on the adapter means (1b, 2b, 3) for connection with a container containing a fluid to be filtered.

11. The filtering device according to claim 1, wherein a support element for supporting the filtering means (8) is disposed at least partially in the area of the bore (5a) of the second plate-like element (5).

12. A method for filtering of a fluid, comprising the steps of:
disposing a dispensing opening of a container containing the fluid to be filtered on the adapter means (1b, 2b, 3) of the filtering device of claim 1; and
actuating a dispensing means of the container so as to press a single dose of the fluid contained therein into the filtering device.

13. The method according to claim 12, wherein the single dose comprises a volume of less than 25 mL.

14. The method according to claim 12, wherein the container comprises a nasal spray having a nasal-adapter, a syringe having a Luer's cone or a MDI having a valve tube (10), and wherein the nasal-adapter, Luer's cone or valve tube, respectively, of the container is disposed on the adapter means (1b, 2b).

15. The method according to claim 14, wherein the adapter means (1b, 2b, 3) comprises a recess (3b) having a shape corresponding to the nasal adapter, Luer's cone or valve tube (10), respectively, and wherein the nasal adapter, Luer's cone or valve tube (10), respectively, is disposed in the recess (3b) of the adapter means (1b, 2b, 3).

16. (canceled)
